(19) United States
(12) Patent Application Publication
(10) Pub. No.: US 2016/0122799 A1
(43) Pub. Date: May 5, 2016

(54) Fecal Recovery Assay

(71) Applicant: Pfizer Inc., New York, NY (US)

(72) Inventors: Julie McKinney, Charles City, VA (US); Lori Lathrop Stern, Woodbine, MD (US)

(73) Assignee: Pfizer Inc., New York, NY (US)

(21) Appl. No.: 14/873,424

(22) Filed: Oct. 2, 2015

Related U.S. Application Data
(60) Provisional application No. 62/059,272, filed on Oct. 3, 2014.

(51) Int. Cl. C12Q 1/24; C12Q 1/68

(52) U.S. Cl. CPC C12Q 1/24; C12Q 1/689

(57) ABSTRACT

The fecal recovery assay of this invention accurately quantifies the number of viable BB12 colonies in the gastrointestinal tract of humans and animals. This BB12 culture method is highly selective for isolating BB12 colonies from human stool. Furthermore, this method has the utility of assessing the survivability of BB12 delivered by food products and supplements through the GI tract, and thus providing potential information on the human health effects of viable BB12.

### FIELD OF INVENTION

Numerous species of Bifidobacteria bacterial strains are used as probiotics in health-promoting dairy products and dietary supplements and one of the most popular probiotic Bifidobacteria strains is B. animalis subsp. lactis (BB12). BB12 colonies can be accurately identified based on morphology from stool culture in modified Wilkins-Chalgren media with added tetracycline using previously described PCR methods. However, those culture-dependent methods are unreliable for the identification and enumeration of Bifidobactera species. The fecal recovery assay of this invention accurately quantifies the number of viable BB12 colonies in the gastrointestinal tract of humans and animals. This BB12 culture method is highly selective for isolating BB12 colonies from human stool. Furthermore, this method has the utility of assessing the survivability of BB12 delivered by food products and supplements through the GI tract, and thus providing potential information on the human health effects of viable BB12.

### BACKGROUND OF THE INVENTION

Bifidobacteria are Gram-positive, bifid-shaped anaerobes that play a pivotal role in maintaining the intestinal microbial balance of healthy humans and animals. Numerous species of Bifidobacteria have been shown to have multiple health benefits, and are frequently used as probiotics in health-promoting dairy products and dietary supplements. One of the most popular probiotic Bifidobacteria strains is B. animalis subsp. lactis (BB12). BB12 has been shown to be less sensitive to stressful conditions such as bile, acid, and oxygen that can potentially be encountered in the GI tract and in manufacturing processes. These characteristics have made BB12 one of the most widely used probiotics in dairy products and supplements in North America and Europe. BB12 is also one the most studied probiotic strains, and has shown efficacy in prevention of traveler's diarrhea, treatment of viral diarrhea, modulation of intestinal flora, improvement of constipation, modulation of immune response, and alleviation of atopic dermatitis symptoms in children.

Previous studies have demonstrated that the recovery of the incorporated probiotic organisms, including bifidobacteria, in food products and supplements is often poor. As the beneficial effects of probiotics, and BB12 in particular, have become increasingly well-established and their popularity and use becomes more widespread, there is a greater need for accurate identification and quantification of probiotics to test the quality and utility of these products. There are established culture-independent methods for identifying and enumerating Bifidobacteria species using 16S ribosomal gene variable regions. Identification of a highly conserved single-copy tuf gene encoding the elongation factor Tu involved in bacterial protein biosynthesis provided a unique marker that could differentiate BB12 from other Bifidobacteria species, and has been used to develop a highly specific quantitative PCR assay. However, PCR-based assays do not demonstrate the presence of viable colonies. While dead bacteria may have impact, it is thought that the majority of the health benefits associated with probiotics is given by viable bacteria.

On the other hand, culture-dependent methods have been found to be insufficiently selective for Bifidobacteria species and inaccurate for producing quantitative results. This issue highlights a need for the development of suitable culture-dependent methods that can demonstrate the presence of viable bacteria.

### SUMMARY OF THE INVENTION

The fecal recovery assay of this invention accurately quantifies the number of viable BB12 colonies in the gastrointestinal tract of humans and animals. This BB12 culture method is highly selective for isolating BB12 colonies from human stool. Furthermore, this method has the utility of assessing the survivability of BB12 delivered by food products and supplements through the GI tract, and thus providing potential information on the human health effects of viable BB12. In the present invention, a novel and selective culture-dependent method for isolating BB12 in human stool was developed.

### DESCRIPTION OF THE DRAWINGS

FIG. 1: BB12 colonies with characteristic fried egg morphology. The magnification is 5×.

FIG. 2: PCR using tuf gene primers on DNA extracted from bacterial colonies: PCR was performed on DNA extracted from putative BB12 (S1) and putative non-BB12 (S2) colonies. Amplicons were resolved on a 1.25% agarose gel, then visualized with ethidium bromide under UV light. MW=100 bp ladder. “Pos” is a positive control; “neg” is a negative control, “H2O” is the water control. The expected size of the tuf gene target BB12 sequence is 117 base, pairs.

FIG. 3: CFU of LGG compared to copies of LGG DNA as determined by qPCR following extraction of DNA from LGG grown to log-phase in broth culture and then serially diluted. Mean data from three independent experiments are shown, with standard deviations. R²=0.99.

FIG. 4: LOG and BB12 colony counts increase after 21 days of supplementation. The solid bars represent the baseline colony counts at the randomization timepoint. The hatched bars represent the colony counts at 21 days after randomization. The red lines represent the lower limits of detection. **p<0.001, *p=0.006

### DETAILED DESCRIPTION OF THE INVENTION

The present invention provides a novel and selective culture-dependent method for isolating BB12 in human stool. Tetracycline was added to Wilkins-Chaigren media modified with acetic acid and mupirocin (WCBM), a media that had been previously demonstrated to be selective for Bifidobacteria in stool culture. Human stool samples were spiked with broth containing BB12 organisms, and cultured on WCBM agar with and without added tetracycline. The accuracy of selection of BB12 from the stool culture was investigated by using tuf gene PCR to confirm the identity of colonies judged to be BB12 based on morphology. Ex-vivo validation was confirmed by quantitatively evaluating BB12 in the feces of volunteers fed known amounts of BB12 in a probiotic mixture also containing Lactobacillus rhamnosus GG.

Enumeration is defined in microbiology is the determination of the number of individual viable microbes in a sample using different techniques.

While the invention has been described with respect to certain specific embodiments, it will be appreciated that many modifications and changes may be made by those skilled in the art without departing from the invention. It is intended, therefore, by the appended claims to cover all such modifications and changes as may fall within the true spirit and scope of the invention.

### EXAMPLES

### Example 1

### Identification and Quantification of Bifidobacterium Animal's Subspecies Lactis Strain BB12 in Human Feces

(a) Preparation of Wilkins-Chalgren Media with Added Antibiotics

Wilkins-Chalgren for Bifidobacterium Muciprocin agar (WCBM) has the following composition: 48 g/L of dehydrated Wilkins-Chalgren Agar (Difco, Detroit, Mich.), 10 g/L glucose, 5 g Agar (Difco), 5 mUL of Tween 80 (Sigma, St. Louis, Mo.), 0.5 g/L of L-Cysteine HCL (Sigma), and 50 mg/L Mupirocin (Sigma). WCBM with tetracycline (WCBMT) has the same composition as WCBM with added Tetracycline (TET). TET was added to achieve final concentrations of 4, 8, 10, 12, and 16 μg/mL in the agar plates.

### (b) Microbiological Assay

A working culture of B. animalis susp lactis (BB12) was streaked for isolation on WCBM and incubated in anaerobic chamber at 37° C. for 72 hours. Inoculum for all testing was prepared by introduction of a single BB12 colony into 25 mL of pre-reduced MRS broth (Difco, Detroit, Mich.). The culture was incubated overnight until an OD600 of approximately 1.0 was reached.

### (c) Spiking Stool

1.0 gram of homogenized stool (confirmed non-probiotic-containing) was suspended in 9 mL of sterile PBS, 200 μl of the overnight broth culture was added to obtain a final spiking inoculum of approximately 10⁷ CFU/g of BB12. Quantitative culture of the spiked stool suspension and the spiking inoculum were performed by preparing 8 1:10 serial dilutions using sterile PBS.

### (d) Plating for CFU Counting and Selection of Potential BB12 Colonies for PCR Assessment

100 μl each of the 10⁻¹ to 10⁻⁹ BB12 spiked-stool dilutions was transferred to the surface of WCBM or WCBMT agar plates. After spreading, the plates were incubated in an anaerobic chamber for 72 hours at 37° C. The plates were then removed from incubation and colonies with “fried-egg”-like morphology, which was unique to BB12 compared to other bifidobacteria species, were enumerated. The CFU/g of stool was then calculated and compared to the amount of BB12 used to spike the stool.

### (e) Preparation of Template DNA

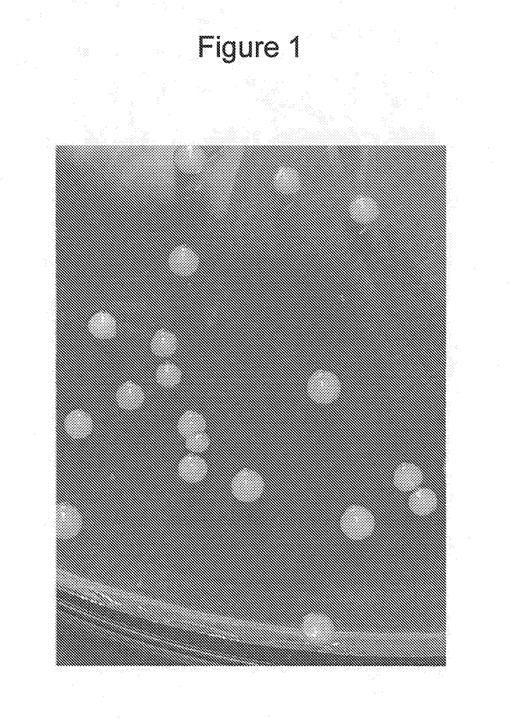

Colonies with the “fried-egg” morphology (FIG. 1) were picked to confirm their identification as BB12 using tuf-gene targeted PCR as follows: Ten individual colonies were picked from a set of agar plates, re-isolated onto WCBM agar and incubated for 72 hours. Individual colonies were then picked and suspended in 100 μL Elution Buffer (Qiagen, Valencia, Calif.). The suspension was boiled for 10 min at 100° C. The crude extraction was stored at −20° C. for future use as template DNA.

### (f) Colony PCR Amplification

PCR mixture contained a final concentration of 666 nmol/L each of forward primer GTG TCG AGC GCG GCA A-3′ and reverse primer 3′-CTC GCA CTC ATC CAT CTG CTT-5′ (17). Thermal profile was as follows; 95° C. for 15 minutes initially, then 30 cycles of the following: 95° C. for 1 min, 65° C. for 1 min, 72° C. for 1 min. All samples were run in triplicate. Positive and negative controls were included in each run. Reaction mixtures were resolved on agarose gels to determine the presence or absence bands of appropriate size (762 bp for LGG and 117 bp for BB-12). In our study, the identification of morphologically chosen colonies was confirmed as positive for BB12 if all three tuf gene PCR triplicates produced amplicons consistent with the expected size of BB12 colonies (˜117 bp), and negative controls did not produce amplicons

(g) DNA Extraction from Broth

Broth culture of BB12 was grown from lyophilized BB12. The broth, along with four serial dilutions of the broth, were DNA extracted using a QiaAMP DNA Stool MiniKit (Qiagen, Valencia, Calif.) according to the manufacturer's instructions with the following modification: samples were reconstituted in 1.4 ml Qiagen ASL buffer to which was added 50.1 mg lysozyme (Sigma, St. Louis, Mo.), 13.5U lysostaphin (Sigma), and 13 μL UltraClean PCR water (MoBio, Carlsbad, Calif.). Following 10 minutes incubation at 37° C., 20 mg of RNAse A (Sigma), 200 mg proteinase K (Qiagen), and 5 mg SDS (Sigma) were added to each tube and tubes were incubated at 70° C. for 10 min, followed by 5 minutes of bead beating in a vortex adapter with 0.5 g of 0.1 mm zirconium/silica beads at 4° C. After removal of beads and insoluble debris by centrifugation, the supernatant was treated to the manufacturer's protocol. The extract was stored at −70° C. until the time of qPCR analysis. This was done in triplicate, and mean data from three independent experiments was calculated.

(h) qPCR Assay

The qPCR mixture contained 500 nmol/L of forward and reverse primers as described for BB-12 colony PCR above, with 250 nmol/L of FAM-labeled probe ATC AAC ACG AAC GTC GAG. Thermal profile was as follows: 5 minutes of initial denaturation at 95° C., followed by 40 cycles of 15 seconds at 95° C., 60 seconds at 60° C., and 30 seconds at 72° C., with fluorescence measured at the end of each annealing step.

A standard curve using a plasmid with the appropriate target sequence was used in each run. For LGG and BB-12, the standard curve ranged in 10-fold dilutions from 5×10⁷ copies of plasmid per 5 μL to 50 copies of plasmid per 5 μL. Each standard was run in triplicate, and the average Ct of each triplicate was determined, and used to create a line of best fit.

R² values ranged from 0.995-0.999. Standard deviation of standard triplicates ranged from 0.0023-1.347. Unknown samples were run in triplicate, and Ct averaged. Standard deviation of unknown samples ranged from 0-1.518. The concentration in each unknown sample was calculated from the line of best fit.

### (i) BB12 Copies vs CFU

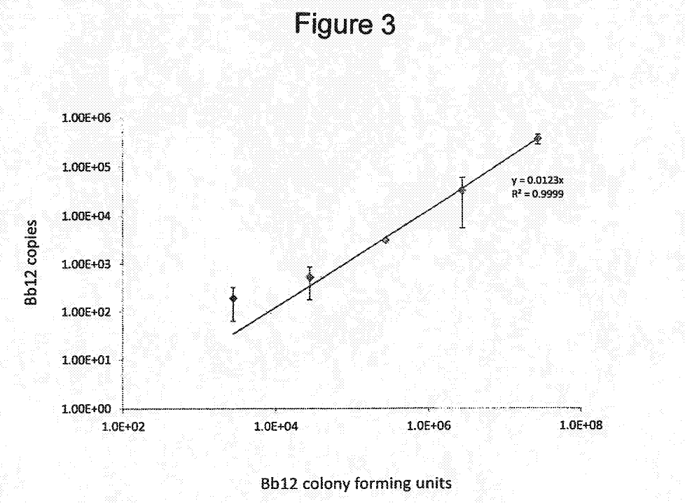

BB12 was grown to mid-log phase in broth culture, diluted, and DNA was extracted from the culture and the first 4 dilutions. Colonies were counted by plating appropriate dilutions on appropriate media. This was performed in triplicate. DNA was extracted from culture and dilutions, then was subjected to qPCR, with each individual sample in a given set tested in triplicate qPCR wells. Number of DNA copies for each unknown sample triplicate was calculated from standard best-fit curves. Triplicate sample copy numbers in each qPCR run were averaged. Results are shown in FIG. 3.

### (j) Results

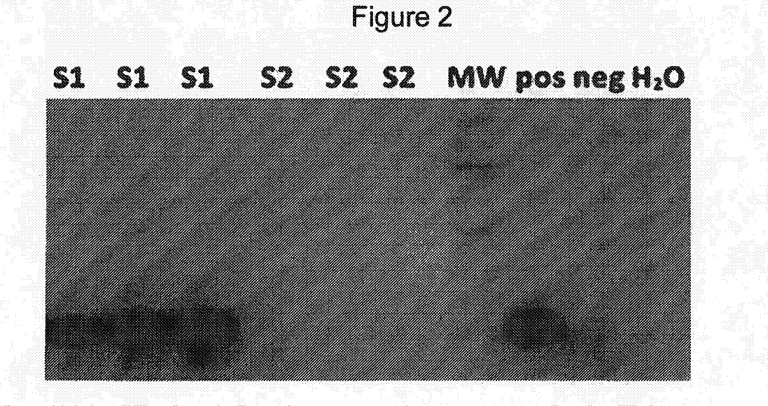

Plating the stool culture on WCBM plates yielded plates crowded with colonies, and it was difficult to distinguish colonies based on morphology. When combining the data of the 7 runs of colonies selected as possible BB12 colonies, only 55/85 (64%) of the colonies were confirmed to be BB12 using tuf gene target PCR (see a representative PCR in FIG. 2). Furthermore when calculating the recovery of the BB12 in the cultures by multiplying the morphology-based count of BB12 by the percentage of colonies confirmed positive by tuf gene PCR, only two of the seven runs gave counts that were within a half log of the amount of BB12 used to spike the stool.

The addition of tetracycline to the agar led to a reduced size and number of colonies on the plate, which allowed for easier identification of colonies that gave the “fried egg” morphology attributable to BB12 colonies (FIG. 1). This allowed for increased selectivity of BB12 on WCBM+tetracycline plates, compared to WCBM alone. The results from five BB12 spiked stools cultured on WCBM agar containing different amounts of tetracycline (one control plate with no tetracycline was also run) are shown in Table 1.

TABLE 1

| | | | | PCR Confirmed Count | | |
|---|---|---|---|---|---|---|
| | | | % | (% concordant X | Log₁₀ Spike − | |
| | Inoculum | Morphology | Concordant | morphology | Log₁₀ PCR | % of Spike |
| Medium | of spike | Based Count | with PCR | based count) | Confirmed Count | Recovered |
| WCBM | 7.90 | 8.91 | 13.3% | 8.03 | −0.13 | 102% |
| WCBMT | 7.81 | 7.56 | 100% | 7.56 | 0.26 | 97% |
| 4 mg/ml | | | | | | |
| WCBMT | 7.55 | 7.72 | 100% | 7.72 | −0.17 | 102.% |
| 8 mg/ml | | | | | | |
| WCBMT | 6.98 | 7.28 | 100% | 7.28 | −0.30 | 104.% |
| 10 mg/ml | | | | | | |
| WCBMT | 6.59 | 5.90 | 85.7% | 5.84 | 0.76 | 89% |
| 12 mg/ml | | | | | | |
| WCBMT | no | N/A | N/A | 0 | 0 | 0% |
| 16 mg/ml | growth | | | | | |

For doses of 4 μg/ml, 8 μg/ml, and 10 μg/ml the lab was able to select colonies that were confirmed to be BB12 by PCR 100% of the time, and for doses of 12 μg/mL they were able to successfully select BB12 85.7% of the time. The 16 μg/ml dose of tetracycline, however, resulted in limited growth of all bacteria species. When compared to the WCBM control plates, which only had successful BB12 selection 13.3% of the time, using plates with added tetracycline significantly increased the ability of the lab to select BB12 colonies based on morphology. Furthermore doses of 4 μg/ml, 8 μg/ml, and 10 μg/ml gave adequate recovery that was within ½ log of the amount of BB12 used to spike the stool.

Further BB12 spiked cultures on WCBM plates with 8 μg/ml tetracycline dose proved to have increased selectivity compared to WCBM, with 94% of selected colonies confirmed to be BB12 by PCR (Table 2). These cultures all had recovery within ½ log of the amount of BB12 used to spike the stool.

TABLE 2

| | | | | PCR Confirmed count | | |
|---|---|---|---|---|---|---|
| | | | % | (% concordant X | Log₁₀ Spike − | |
| Run | Inoculum | Morphology | Concordant | morphology | Log₁₀ PCR | % of spike |
| Reference | of Spike | based count | with PCR | based count) | confirmed count | recovered |
| 1 | 7.00 | 6.58 | 91.70% | 6.54 | 0.46 | 93% |
| 2 | 7.00 | 6.94 | 83.30% | 6.86 | 0.14 | 98% |
| 3 | 7.60 | 8.18 | 100% | 8.18 | −0.30 | 107% |
| 4 | 7.60 | 8.09 | 100% | 8.09 | −0.21 | 106% |

In order to determine the relationship between BB12 and tuf gene copy number and BB12 colony forming unit, a qPCR was performed on DNA extracted from BB12 broth cultures and compared with CFU using methods outlined with WCBM and TET agar above. Quantitative PCR detection of serial dilutions from the DNA of pure BB12 culture was linear for bacterial copy numbers over a range of 1000 to 10,000,000 CFU (FIG. 3). Quantitative PCR detection of serial dilutions of DNA from stool spiked with known amount of BB12 was linear for bacterial copy numbers over a range of 100,000 to 10,000,000 CFU (data not shown).

### Example 2

### Randomized Clinical Trial of the Recovery of Probiotics Lactobacillus rhamnosus GG (LGG) and Bifidobacterium animalis Subspecies Lactis (BB-12) from the Gastrointestinal (GI) Tract of Healthy Volunteers

Probiotics are live organisms that confer a health benefit when administered in adequate amounts to the host. Pfizer Consumer Healthcare (PCH) has developed the dietary supplement ProNutrients® Probiotic containing the probiotics Lactobacillus rhamnosus GG (LGG) and Bifidobacterium animalis subspecies lactis BB-12 (BB-12) in a powder sachet. These organisms have been shown to survive the conditions of the gastrointestinal (GI) tract. This study were confirmed the survival and recovery of the organisms from the GI tract of healthy volunteers after ingestion of the supplement and to assess safety.

(a) Methods: This was a 2 arm, parallel group, open label randomized prospective study of healthy volunteers. All subjects underwent a run-in period (>14 d). Thereafter, the subjects were randomized to a non-supplemented control group (C) or the probiotic (P) group, which consumed 1 sachet of supplement (S) containing >1=1×10⁹ colony forming units (CFU) of each organism/day for 21 d. There was a 28 d post-S period for each group. Fecal samples were collected at baseline (the end of the run-in period), and after 14, 21, and 28 d. LGG and BB-12 were quantified by (1) quantitative culture (qCX) with colony ID confirmed by polymerase chain reaction (PCR) and (2) qPCR. The primary endpoint was the recovery of LGG and BB-12 (CFU/g) from the fecal samples of all subjects at 21 d. CFU were log₁₀ transformed. Paired t-tests were used to compare within subjects change from baseline. ANOVA models with a treatment term were used to In this study, the post-supplementation recovery and survival of the probiotic bacteria LGG and BB-12 in fecal samples from healthy human subjects after 21 consecutive days of consumption of the probiotics within the product matrix, a powder sachet was investigated.

(b) Randomization Time Point (baseline levels): Enumeration of LGG and BB-12 in fecal specimens
(i) Quantitative Culture: One subject had LGG detected at a level of log CFU/g=5.8. The lower limit of detection [LLD] is 3.4. No BB-12 was detected by culture in the subjects.
(ii) qPCR: No subject had LGG detected by qPCR. Three subjects had BB-12 detected and the log copies/g were 5.1, 5.1 and 4.4 with an LLD=4.3. One of those three subjects received no supplementation; however, was also detected by PCR at 14 and 21 days after randomization. BB12 was not detected but not in this subject's sample corresponding to 4 weeks after the end of the supplementation period.

(c) 14 and 21 Day Time Points after Randomization: Enumeration of LGG and BB-12 in fecal specimens

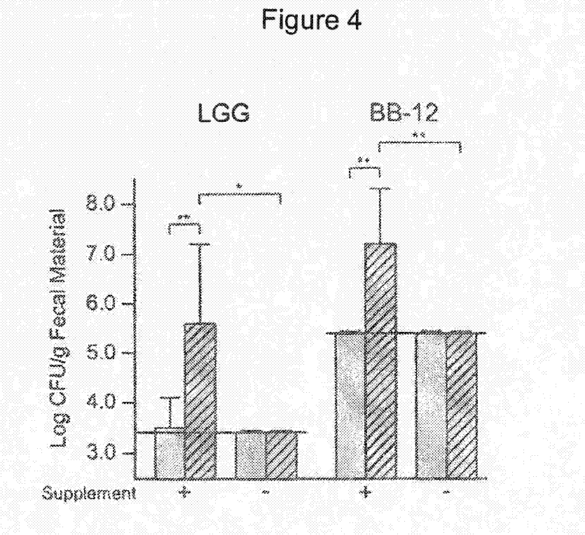

FIG. 4 shows results for recovery of LGG and BB-12 by quantitative culture after 21 days of supplementation. Results similar to FIG. 2 were also obtained by enumeration of LGG and BB12 by quantitative culture at the 14 day time point after randomization. Similar results were also obtained for recovery by qPCR at 14 days and 21 days. The data in FIG. 4 shows that LGG and BB-12 colony counts increased after 21 days of supplementation.

(d) Four weeks Post-Supplementation Time Point: Enumeration of LGG and BB-12 in fecal specimens
(i) Quantitative culture: No subject, regardless of group, had detectable levels of LGG or BB-12 in the stool.
(ii) qPCR: One subject in the no supplement group had LGG detected by qPCR (log copies/g=4.8, lower limit of detection 4.3). Three additional subjects who received supplement had BB-12 detected by qPCR (log copies/g of 4.7, 5.1 and 5.9, lower limit of detection 4.3).

LGG was infrequently detected in the GI tract of healthy individuals in the absence of supplementation, suggesting that it is not common in the environment. Both LGG and BB-12 in viable form were recovered from the GI tract of healthy individuals after 14 and 21 days of supplementation. This result suggests that ingestion of the supplement for at least 14 days results in the delivery of live organisms which can then survive in the GI tract of healthy persons. There was clearance of viable LGG and BB-12 within 4 weeks after cessation of supplementation. It is possible that the detection of BB-12 by qPCR 4 weeks after the cessation of supplementation was a result of residual DNA in the absence of viable organisms in the GI tract.

The BB12 culture method of the invention was shown to be highly selective for isolating BB12 colonies from human stool. In contrast to previous studies that have found culture-dependent methods to be unreliable for the identification and enumeration of Bifidobactera species, this novel method can be used accurately to quantify the number of viable BB12 colonies in the gastrointestinal tract of humans and animals. This method has the utility of assessing the survivability of BB12 delivered by food products and supplements through the GI tract, and thus providing potential information on the human health effects of viable BB12.

1. A method for measuring recovery or survival of probiotic organisms in human fecal samples from the gastrointestinal tract of a subject.

1. The method of claim 1 wherein the probiotic organisms in the human fecal samples are isolated and enumerated.

2. The method of claim 1 wherein the probiotic organisms that are isolated and enumerated are selected from Bifidobacterium animalis, subspecies lactis BB12 and Lactobacillus rhamnosus GG.

3. The method of claim 1 wherein the Bifidobacterium animalis, subspecies lactis BB12 and Lactobacillus rhamnosus GG are enumerated using quantitative culture confirmed by PCR.

4. The method of claim 1 wherein the Bifidobacterium animalis, subspecies lactis BB12 and Lactobacillus rhamnosus GG are enumerated using quantitative PCR.

5. The method of claim 1 wherein the Bifidobacterium animalis, subspecies lactis BB12 is isolated in fecal samples by culture on MRS agar with added tetracycline.


SEQUENCE LISTING (3 sequences)

SEQ ID NO: 1 - DNA, 16 bp, Artificial Sequence
  Synthetic Construct
gtgtcgagcgcggcaa

SEQ ID NO: 2 - DNA, 21 bp, Artificial Sequence
  Synthetic Construct
ctcgcactcatccatctgctt

SEQ ID NO: 3 - DNA, 18 bp, Artificial Sequence
  Synthetic Construct
atcaacacgaacgtcgag